(19) United States
(12) Patent Application Publication
(10) Pub. No.: US 2005/0107723 A1
(43) Pub. Date: May 19, 2005

(54) Methods and apparatus for determining work performed by an individual from measured physiological parameters

(76) Inventors: Thomas C. Wehman, Cupertino, CA (US); Serjan D. Nikolic, San Francisco, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 10/779,149

(22) Filed: Feb. 12, 2004

Related U.S. Application Data
(60) Provisional application No. 60/447,968, filed on Feb. 15, 2003.

(51) Int. Cl. A61B 5/103

(52) U.S. Cl. 600/595; 128/920

(57) ABSTRACT

Methods and apparatus for gathering and processing data sensed on an individual from portable heart monitors and accelerometers aligned along three orthogonal axes determine substantially equivalent oxygen consumption information during an individual's physical activities without requiring gas-flow or gas-analysis equipment. Such information promotes calculations of physiological energy expenditures, and analyses of the accelerometer data associated with a specific sensing location on an individual's body provide indication of the particular physical activity for selecting appropriate scaling factors and filtering requirements in analyzing the data to determine various parameters indicative of the individual's expenditure of physiological energy, and other health-oriented factors.

### RELATED CASES

This application claims priority benefit from provisional application Ser. No. 60/447,968 entitled “Method And Algorythem For Treating Measured Physilogical Parameters To Determine Work Performed By An Individual”, filed on Feb. 15, 2003 by Thomas Clifford Wehman and Serjan D. Nikolic. The subject matter of this application relates to the subject matter of U.S. Pat. No. 6,436,052 entitled “Method and System for Sensing Activity and Measuring Work Performed by an Individual,” issued on Aug. 20, 2002 to S. Nikolic, et al., which subject matter is incorporated herein in its entirety by this reference to form a part hereof.

### FIELD OF THE INVENTION

This invention relates to methods and apparatus for physiological monitoring of an individual during various physical activities, for example, for determining the amount of work performed by an individual during such activities, or for providing indicia of the individual's heath condition.

### BACKGROUND OF THE INVENTION

Human health condition can be determined and treated upon analyzing specific physiological characteristics of a human body. The rate at which the human body consumes oxygen provides a reliable measurement for analysis of work performed by the human body. Within the body, the cardiovascular system delivers oxygen to the muscles for the use in oxidizing various fuels such as carbohydrates and fats to yield energy. This rate of oxygen consumption is commonly known as VO₂ and, when compared to cardiac response, provides an indication of the health of the individual's cardiovascular system.

Traditionally, an individual's VO₂ has been obtained by comparing the individual's inhaled air volume with exhaled air volume. This comparison is performed on air volumes measured while the individual is connected to a gas analyzer and runs on a treadmill in a specialized testing facility.

Other measures of a body's physiological activity include Heart Rate (HR), calorie (C) expenditure, and METS, or multiples of an individual's energy consumption at rest. Heart rate is a measure of how many times a heart beats in a minute, and decreases or increases during physical activity or mental stimulation. Calorie expenditure is actually Kilocalorie expenditure, but by medical convention is oftentimes referred to simply as calorie expenditure as a measure of biological energy consumption. A MET is a metabolic equivalent and is usually defined as the energy equivalent of 1 Kcal/Kg/hour, or about 3.5 ml/Kg/min (VO₂).

While the rate of oxygen consumption provides valuable information for determining an individual's fitness, the traditional method for measuring VO₂ is very confining and does not allow the individual to perform usual physical activities under normal environmental conditions.

It would therefore be desirable to determine an individual's rate of oxygen consumption, maximum rate of oxygen consumption, heart rate, calorie expenditure and METS during physical activity in a location where that physical activity would normally take place, (i.e., in Free Space) rather than in a specialized testing facility. Further, it would be highly desirable to be able to determine an individual's rate of oxygen consumption during a normal physical activity without actually measuring the gas flows with cumbersome attached equipment.

It would also be desirable to display information about the health of an individual's cardiovascular system on a real time basis, and be able to download such information to a central station for further analysis and archiving. It would also be desirable to simultaneously monitor several individuals as they perform various activities in order to establish ‘average’ baseline parameters for each individual or, for example, from among a group of healthy, well-conditioned athletes. This promotes comparisons in real time of current levels of energy expenditure and body response to a previous session of activity, or to a baseline activity energy expenditure, or to reference levels of “normal, healthy individual” responses for certain activities.

### SUMMARY OF THE INVENTION

The present invention determines an individual's rate of oxygen consumption and maximum rate of oxygen consumption without measuring actual gas flows, and also measures heart rate, for determining calorie expenditure and METS in order to measure the amount of work performed by the individual's body. Heart rate, and acceleration along multiple axes, are measured and stored in a local storage device for analyses and display in real time, and optionally for download to a local base station. After the local storage device or the base station receives the outputs, the heart monitor and accelerometer are available to take additional measurements in successive time intervals. The base station may upload data and analyses to a central clearinghouse for processing. More specifically, the acceleration outputs are collected and processed to initially convert the outputs into motion information and then into activity information. The heart rate and activity information may then be graphed on the same or similar time base for determining their relationships in order to calculate cardiovascular response to the activity. Comparison to previous activity sessions, or to base line energy expenditure, or to reference “normal, healthy” responses from certain populations can be made and displayed substantially in real time. A cardiovascular index (CI) or similar index may be calculated by dividing the total amount of work or energy expended by the total number of heart beats during a period of time that both the energy and the heart rate are monitored.

The apparatus of the present invention determines an individual's rate of oxygen consumption, maximum rate of oxygen consumption, heart rate and calorie expenditure in order to determine the amount of work performed by the individual's body. This allows heart rate and acceleration measurements to be taken in a ‘free-space’ environment such as in a gymnasium or a swimming pool, on a track, a court, or a field, or at home without requiring traditional gas-flow equipment to facilitate the activity taking place under normal conditions.

### BRIEF DESCRIPTION OF THE DRAWINGS

FIG. 1 is a pictorial illustration of a typical operating environment for the present invention.

FIG. 2A is a block schematic drawing of monitoring apparatus in accordance with one embodiment of the present invention.

FIG. 2B is a schematic diagram of another embodiment of the monitoring apparatus of the present invention.

FIG. 3 is a flow chart illustrating a method for processing the sensed data in accordance with one embodiment of the present invention.

FIGS. 4A, 4B, 4C are graphs illustrating the output data from accelerometers aligned along three axes.

FIG. 5 is a graph illustrating the filtered maximum change in total dynamic acceleration over an interval of time as derived from output data from the accelerometers.

FIG. 6 is a graph illustrating a comparison of a plot of the filtered maximum change in total dynamic acceleration as offset in time from a plot of conventionally-measured VO₂.

FIG. 7 is a graph illustrating heart rate response to acceleration or comparable VO₂ rate for a healthy subject.

FIG. 8 is a graph illustrating heart rate response to acceleration or comparable VO₂ rate for a patient with congestive or chronic heart failure (CHF).

### DETAILED DESCRIPTION OF THE INVENTION

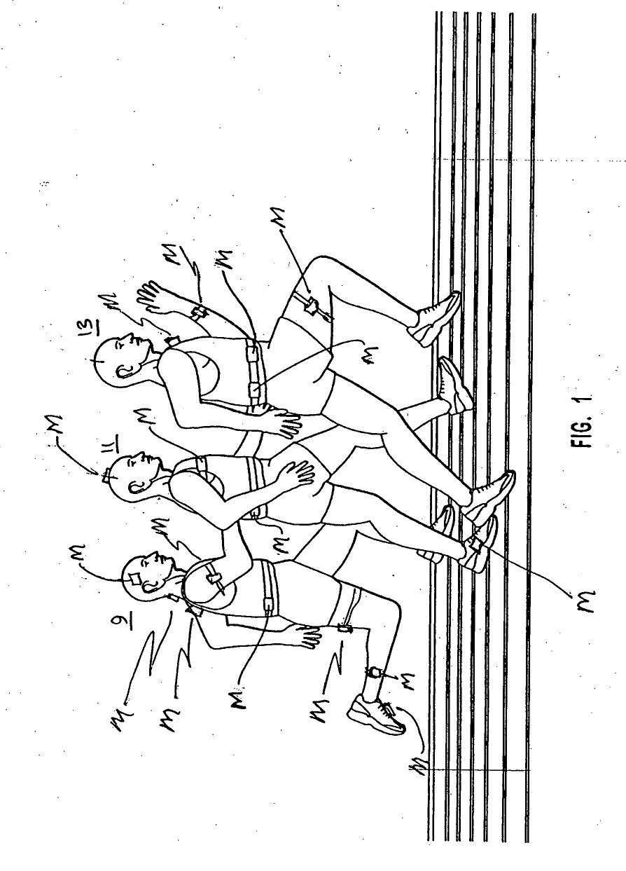

Referring now to FIG. 1, there is shown a pictorial illustration of a typical ‘free space’ environment in which individuals 9, 11, 13 may be fitted with monitoring devices M during a physical activity such as sprint-running or related competitive track events. Alternatively, a device may be embedded subcutaneously on an individual. It is desirable to determine each individual's rate of oxygen consumption (i.e., VO₂) and maximum oxygen consumption without hampering physical performance with traditional gas-flow equipment attached to the individual. In addition, it is desirable to determine total calories expended, heart rate and total METs in order to determine the amount of work performed by the individual's body. In accordance with the present invention, these parameters are determined during the physical activity in a location where the physical activity would normally take place, such as on a track, a field, a court, in a gymnasium, a swimming pool, or at home. One or more monitors M may be attached to an individual at various bodily locations to measure the individual's heart rate and acceleration during the physical activity. If a heart rate monitor is not available, an estimated heart rate may be calculated from known relationship with physiological responses to acceleration that is monitored along three axes. The measurements are processed to determine the VO₂ for that individual's body and to determine the relationship between the individual's activity and heart rate.

The invention is described below in reference, for example, to calculating the amount of work that is performed by an individual's body through determining the individual's VO₂, or equivalent, during a physical activity, under normal conditions. “Physical activity” refers to any type of exercise, exertion or movement that the individual undergoes during the period of time that measurements are taken, and further includes normal daily activities, whether at nominal rest or in a period of physical exertion. Examples of physical activity include running, walking, jogging, jumping, swimming, biking, pushing, pulling, or any other type of physical movement that a human body can undergo.

“Normal conditions” refers to the surrounding circumstances and manners under which a particular individual undergoes a physical activity during which the measurements are taken. By way of example, “normal conditions” includes performing physical activity on a track, court, field, or a street, on grass, concrete, or carpet, in a gymnasium or swimming pool, at home or at work or any other environment or location where the individual usually undergoes physical activity. Furthermore, “normal conditions” connotes substantial absence of artificial conditions that affect the physical activity being performed by the individual. Of course, the present invention is applicable to determining the VO₂ or work of an athlete as well as for all individuals undergoing recreation or daily routines.

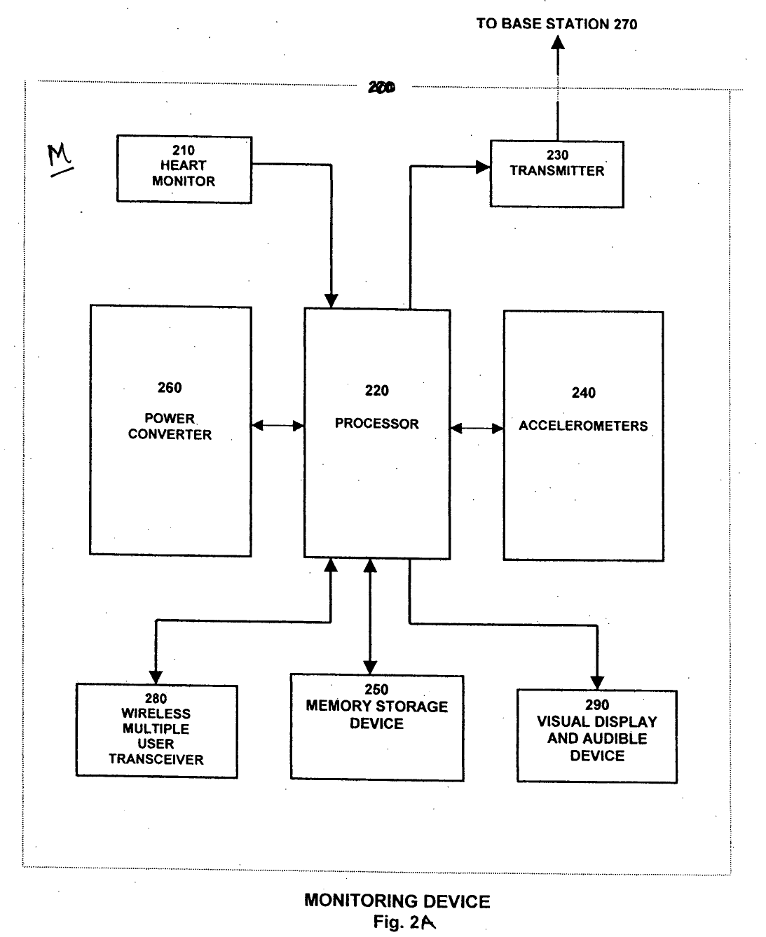

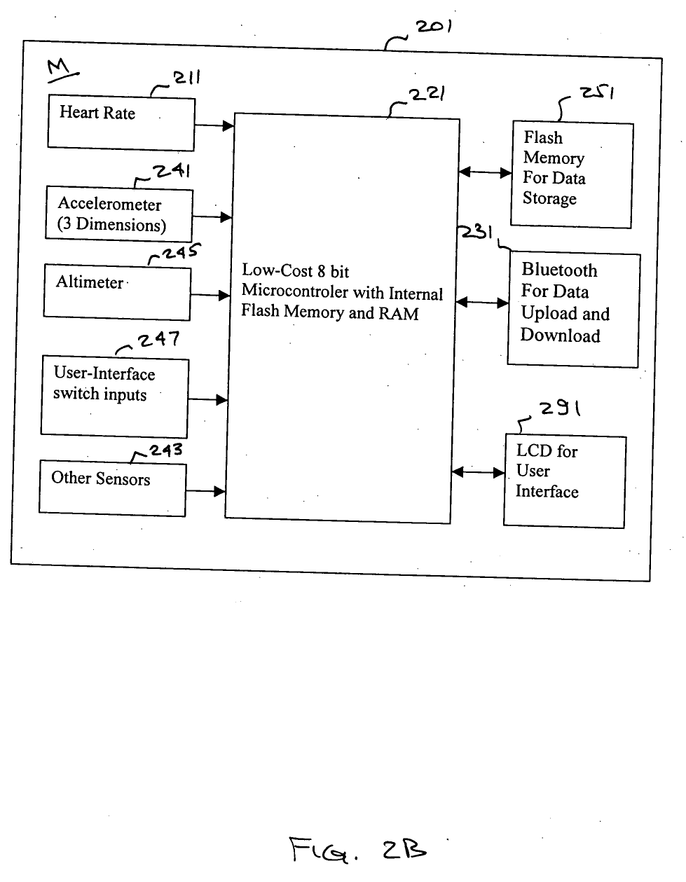

Referring now to FIG. 2A, there is shown a monitoring device, M, as illustrated on an individual 9, 11, 13 in FIG. 1, that includes a heart monitor 210 and accelerometers 240 oriented along three orthogonal axes. The heart monitor 210 may be any type of device that senses heart rate by sound or ECG signals, or the like, and supplies the sensed data to processor 220 that also receives the data from the accelerometers 240, and other forms of monitoring data for digitizing and processing and storing in storage device 250. A power converter 260 including batteries for portable operation powers the processor 220 and other components to facilitate convenient portable use during physical activities of an individual. The processor 220 also controls a transmitter 230 or a transceiver 280 for transferring data to and from a base station 270 (not shown) that operates on the data for one or more individuals in a manner as later described herein. The processor 220 also controls visual display and audible output device 290 for providing sensory feedback to the individual of substantially real time analysis of various monitored and computed parameters indicative of the individual's heart and health conditions. In addition, sensory feedback may be supplied to the individual, for example, in response to a predetermined goal or parameter involving energy expenditure is attained. The wireless transceiver 280 (or transmitter 230) may operate on conventional RF channels, or on contemporary ‘Blue Tooth’ radio telemetry for exchanging data and computed results between each monitoring device 200 and a remote base station 270. Alternatively, the monitoring device 200 may include sufficient computational capability to process the sensed data internally, rather than at a base station 270, for determining such parameters as total VO₂, maximum VO₂, total expended energy, heart rate, and the like, for display on device 290.

Referring now to FIG. 2B, there is shown a block schematic diagram of an embodiment of the monitoring device M shown in FIG. 1. In this embodiment, the monitoring device M (201) includes a microprocessor 221 that may, for example, contain internal memory, operate in 8-bit processing mode, and include analog and digital I/O ports for interfacing with attached sensors and input devices for performing algorithms, as described herein, and for controlling operations of the monitoring device 201. Specifically, such sensors and input devices include heart-rate sensor 211 of the sound-sensing or EGC-sensing (or other) types, and include three accelerometers 241 aligned along orthogonal X (fore and aft) and Y (side to side) and Z (vertical) axes, and other sensors 243, 245 such as thermal and altimeter devices that are sensitive, respectively, to temperature and ambient pressure. Altimeter data is useful for calculating physiological energy expended in uphill and downhill activities, and temperature data is useful for analyzing over exertion of an individual, or ambient temperature conditions. In addition, the microprocessor 221 is connected to the user-interface 247 (e.g., keyboard) for selectively entering data (e.g., individual's mass, proposed activity from a displayed menu of activities, and the like).

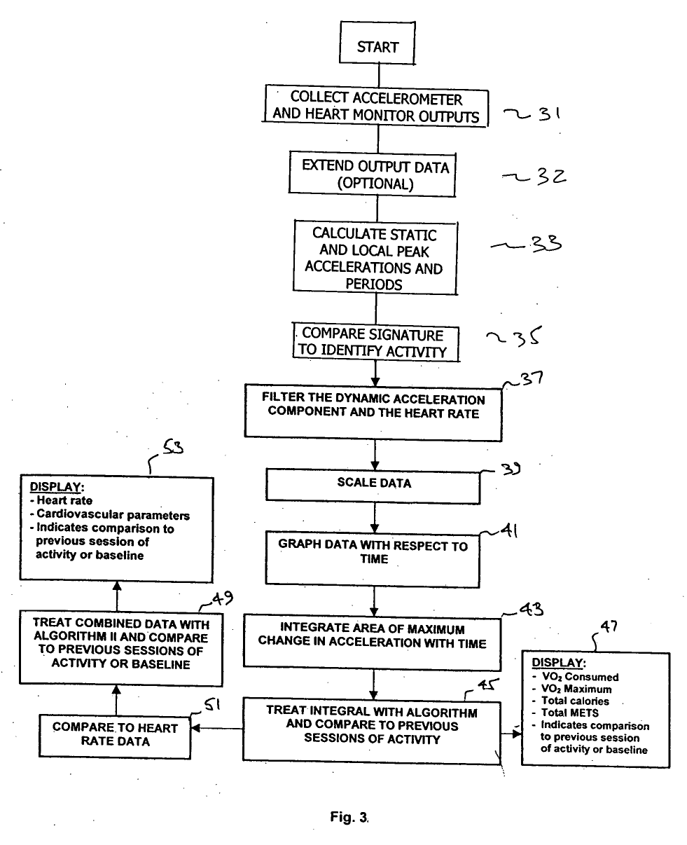

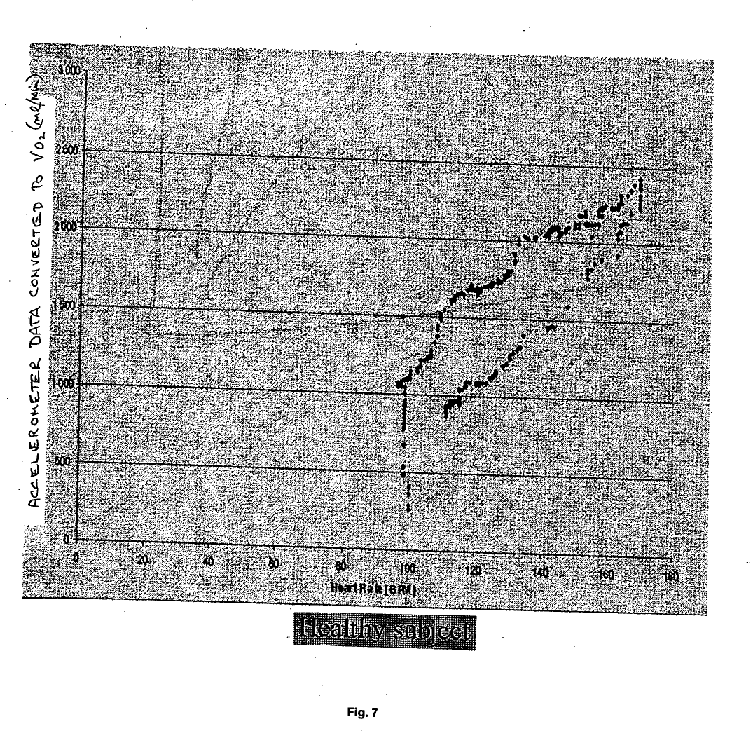

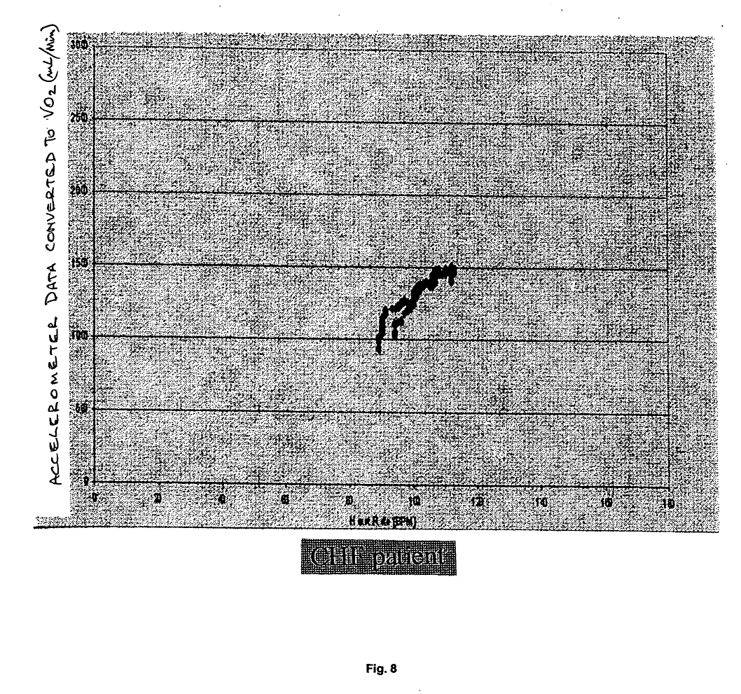

The microprocessor 221 also controls flash memory device 251 for compaction, storage and retrieval of data, and controls of wireless interface 231 such as a ‘Blue Tooth’ RF channel for uploading and downloading data, instructions and remote calculations. In addition, the microprocessor 221 controls an LCD display 291 suitable for indicating data entries, calculations and graphic illustration (e.g., similar to FIGS. 7, 8), all in accordance with operations of the monitoring device 201, for example, as described herein with reference to the flow chart of FIG. 3.

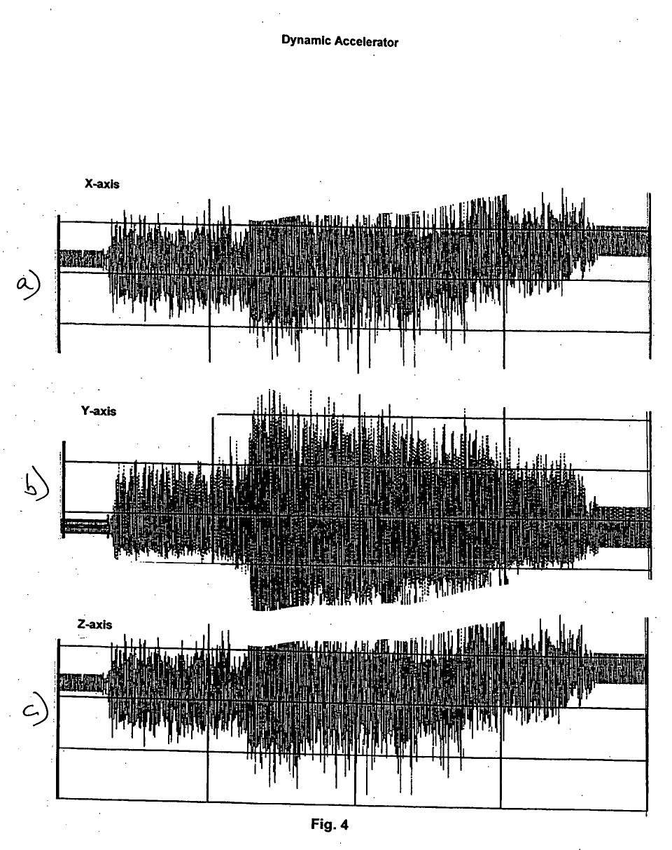

Referring now to the flow chart of FIG. 3, there is shown one embodiment of the method for determining various parameters indicative of an individual's health status. Specifically, various data are collected 31 from the accelerometers 240, 241 aligned along three axes and other data sensors such as the heart monitor 210, 211. The data collected from the accelerometers aligned, for example, along orthogonal axes X, Y, and Z may be in the form as illustrated in FIGS. 4A, 4B, and 4C for a particular attachment location on the body of an individual, for a particular physical activity. Misalignment of the accelerometer axes relative to orientation on an individual's body may be corrected conventionally in the vector analyses for performing energy calculations corrected for angular misalignments. At other attachment locations and during other physical activities, the waveforms produced by each of the accelerometers will vary and provide a ‘signature’ or characteristic waveform. Thus, a monitoring device 200, 201 attached to an individual near the temple during running activity and having an accelerometer aligned along a vertical axis will respond differently, for example, during a running or jumping activity than during a rowing or bicycling activity in which the vertical-axis activity is significantly diminished although the physiological energy expended may be comparable. Thus, analyses 33 of the waveforms from the accelerometers in a monitoring device 200, 201 attached at a particular location on an individual, and attributable to accelerations along the orthogonal X, and Y, and Z axes, thus provide indication of the type of physical activity in which the individual is engaged. Such determination of the physical activity of the individual is useful for properly scaling the data in energy formulas for different activities, as later described herein.

An activity can be selected through the user interface 247 by scrolling through a menu to select the activity in which the individual will engage, or the activity can be determined by the signature of the activity, as described herein. The signature includes average or maximum magnitude, direction, periodicity and changes in one or more of these parameters for each of the three accelerometers 240, 241. Other input components for the signature analysis can also include ambient temperature, heart rate, altimeter for atmospheric pressure (hiking or running up and down hills), and any other endogenous or exogenous factors that may be useful for determining a particular activity, such as chlorine or water pressure detection for pool sports. For example, a rise in the X (forward and reverse) and Z (up and down) magnitudes with regular periodicity might indicate the difference between walking and running. Erratic changes in Y magnitude (sideways or turning motions) with short spurts of X and Z periodicity might indicate basketball activity, or the like.

A matrix of these signatures for various activities are kept in tabular form, and best fits to particular table entries determine a candidate activity. Sometimes correct selection of the particular activity will make little difference (e.g., volleyball and basketball) since both activities may have substantially the same scaling constant in the energy formula.

The data from the heart monitor is time-stamped at each sensed heartbeat, and such data along with accelerometer data may be compressed and stored in the storage device 250, 251 for subsequent downloading via wireless link 230, 231, 280 to a base station 270 having greater computational capability than within the monitoring device 200, 201. Of course, requisite computational capability may be incorporated into the monitoring device 200, 201 along with adequate battery power to accomplish the computational requirements, as described later herein.

For brief intervals of physical activity, it may become desirable to extend 32 the sensed data in order to provide sufficient number of data points to accommodate conventional smoothing algorithms. For example, initial few data points at the start of an activity-monitoring session may be selected and replicated numerous times, for example, as more fully described in the aforecited U.S. patent. Similarly, terminal few data points may be selected and replicated numerous times, as may be needed for proper operation of a conventional smoothing algorithm.

The sensed data may be compacted in the memory device 250 to save space in the memory that can be any read/writable memory such as flash, EEROM disk, and the like. A simple conventional compression scheme is chosen to store as much information as possible on the media involved.

If data is reasonably regular with regard to accelerometer magnitude and periodicity, then only one or few cycles of this data needs to be recorded with a count of the number of such cycles in a manner similar to run-length encoding that is commonly used for repeated data values. For walking, jogging and running this can amount to considerable memory savings since these activities have highly-regular, repeated accelerometer patterns.

Another method to save storage space is to reduce the amount of data collected, for example, by sampling for a short period (e.g. 10 samples per second for 10 seconds), then waiting for a longer period (e.g, 50 seconds) and sampling again to provide a reasonably, accurate indication of the activity.

The method of the present invention develops parameters by which the monitored individual's activity can be identified (e.g., for use in scaling data, as later described herein). The sensed data from the three accelerometers is analyzed 33 for peak or average magnitude and periodity in connection with heart rate. For example, static and dynamic acceleration components (e.g., gravity vs. activity) are segregated from the sensed accelerometer data, and the signature characteristics of such data may be compared 35 with a matrix of known characteristics for a variety of physical activities (e.g., running, bicycling, rowing, and the like), as developed from actual testing. Such matrices may be stored locally in the storage device 250, 251 or, more likely, stored at a remote base station 270 for interoperable computation over wireless communication link 230, 231, 280 with the monitoring device 200, 201. The normalization and benefit of such sensed data then determines the activity involved for establishing appropriate multipliers or coefficients (e.g., scaling factors) to be used with the data in energy calculation formulas, as set forth in the attached Appendices I and II.

Specifically, the dynamic components of the sensed accelerometer data is filtered or smoothed 37 for example, using conventional curve-fitting techniques. In the case of repetitive activities, conventional sinusoidal curve fitting is one suitable technique for smoothing the sensed data from each of the three accelerometers. The sensed heart rate may be filtered 37, for example, using a succession of three or four samples to determine a moving-average value.

Energy calculation may be substantial as disclosed in the aforecited U.S. Pat. No. 6,436,052 with the addition of the third axis accelerometer data. Further, the data may be refined by adding altitude data from altimeter 245. A measure by an altimeter of the atmosphere pressure is made periodically and that information is converted to altitude data. A positive change in altitude represents work or energy expenditure to raise the mass of that individual through that altitude change H. Thus, W=MgH, where M is the mass of the individual and g is the force due to gravity. This result, converted to the appropriate units, is added to the activity formula for each positive elevation change in a course either by bicycle or on foot.

For exercise cycles with variable loads and treadmills with inclines, the load information may be manually entered into computations, or heart rate may be used to infer the load. The percent change in heart rate over the heart rate expected for a given duration on a no-load exercise device, times an appropriate work factor may be added to the formula for energy expenditure. This load information can also be done by using the percent change in heart rate, times a scale factor and using this factor as a base energy formula multiplier in addition to using the constant multiplier for the determined activity.

Thus:
W=αM*Sum(accmag)+λ(ΔHr %) or
W=βαM*Sum(accmag);
where β=φ(ΔHr %);
α is the constant multiplier for the determined activity;
λ is the determined work factor;
φ is a determined scale factor; and
M*Sum(accmag) is the subject's mass times the integral of the accelerometer 3-axis resultant magnitude, as described herein.
   Alternatively, W=αM*Sum(accmag)+μM;
where μM represents the at-rest energy consumption for a body of mass M. The multiplier μ can be different depending on whether the subject is lying down, seated or standing and this can be determined by the direction of the resultant accelerometer vector due to gravity.

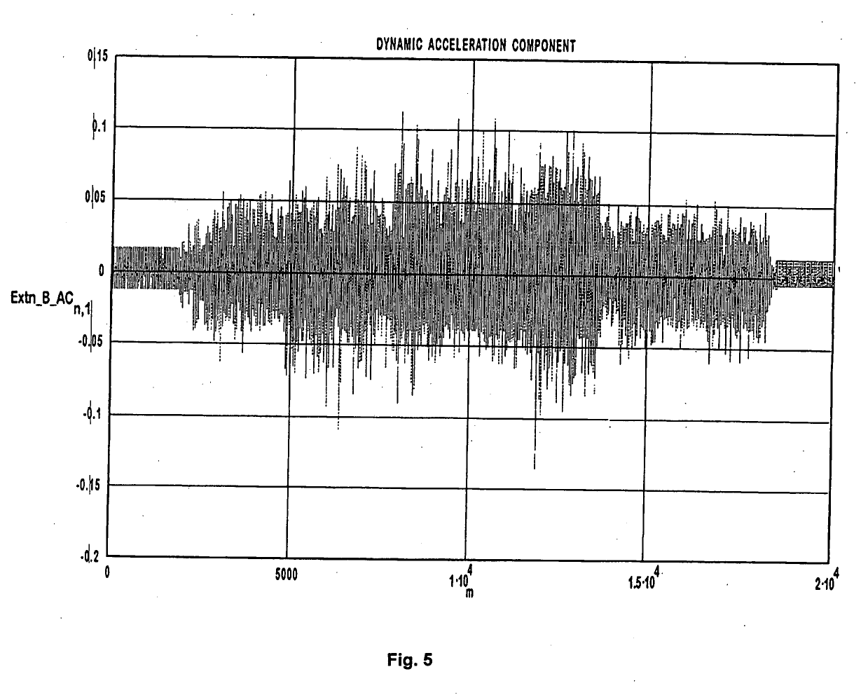

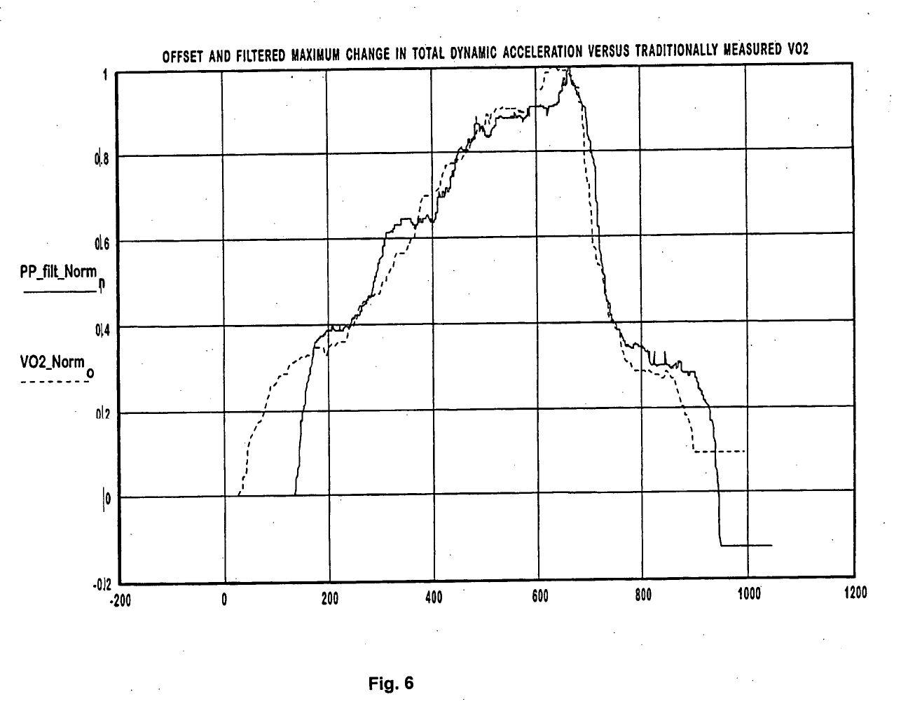

The static or gravitational component of the sensed data from each of the three accelerometers may be scaled 39 into ‘g’ units for use in energy conversion formulas, for example, as set forth in the attached Appendices I and II, and for graphing 41 with time either as individual waveforms (as shown in FIGS. 4A, 4B, 4C) or as a single waveform (as shown in FIG. 5) that represents the vector composite magnitude of the three separate component waveforms. The maximum changes in total dynamic acceleration over the time of the activity may be graphed, as shown in FIG. 6, for comparison with actual gas-flow measurement of VO₂ for closely correlated or equivalent results.

The integral of the resultant or composite accelerometer vector magnitude is achieved 43 by summing these magnitudes over the time of the physical activity. The integrated value is multiplied by a person's mass and the appropriate (or scaled) coefficient for the identified activity to determine the person's energy expenditure in excess of the rest energy expenditure. The resultant can then be normalized or converted to desirable units such as V0₂ consumed, or maximum V0₂, or total calories, or total METS, or the like, for display 47 and comparisons with results of preview performances, or with other suitable baselines. Such comparisons 49 with associated heart rates 51 are useful for displaying 53 cardiovascular characteristics of the individual.

An energy calculation formula, as described in the aforecited U.S. Pat. No. 6,436,052 includes the numeric computation of the integral of the magnitude of the smoothed accelerometer data (g component removed) for a relatively short time span, times a constant (derived as above by recognizing the exercise activity, or stipulated for the given activity). The total energy expenditure is the accumulated sum of these calculated units over the duration of the activity.

Referring now to the graph of FIG. 7 for a healthy individual, there is shown one practical display of the equivalent V0₂ (e.g. in ml/min) derived according to the present invention charted against the individual's heart rate. This chart shows wide dynamic ranges of V0₂ and heart rate over the interval of a physical activity, to maxima achieved for the activity. Following cessation of the activity, the equivalent V0₂ and the heart rate decrease approximately linearly toward rest conditions.

In contrast, an individual suffering chronic or congestive heart failure (CHF) exhibits severely limited ranges of V0₂ and heart rate, as illustrated in the graph of FIG. 8.

Therefore, the methods and apparatus of the present invention provide substantially equivalent indications of rate of oxygen consumption and maximum rate of oxygen consumption using data from portable accelerometers positioned at a selected location on an individual and substantially aligned along three orthogonal axes. Heart rate is monitored for analyzes with the equivalent VO₂ determinations to provide indications of various parameters such as total physiological energy expenditure and cardiopulmonary activity. In addition, analyses of the accelerometer data along three orthogonal axes, oriented about a specific attachment position on an individual's body thus provide ‘signature’ indications of the individual's particular physical activity. Scaling of the accelerometer data for the identified physical activity correlates levels of accelerometer activity along three axes during various physical activities with the equivalent rates of VO₂ consumption for the activity (e.g., during swimming and during walking). Monitoring devices for attachment at various locations on individuals sense various parameters such as heart rate and accelerometer activities for self-contained processing and storage and display of health-oriented parameters. Alternatively, such monitoring devices may transfer data to and from remote stations via conventional wireless communication channels for remote computations and storage of data, including return transfers of calculated results for display via the monitoring device. Such display as audible or visual information may include heart rate, total VO₂, maximum VO₂, calorie expenditure, METS, physiological energy expanded, and the like, that can be calculated and stored for comparison against results determined during prior intervals of a particular physical activity, or against a base-line average of results determined for healthy individuals engaged in such physical activity.

### Appendix I

### Treadmill VO₂ vs. Teem

Definitions:

1. TEEM=Total Energy Expenditure Measurement
2. Acceleration (A)=Distance/Time²=D/T²
3. Force (F)=Mass×Acceleration=M×A
4. Mechanical Work (Wm)=Force×Distance=F×D or by substituting (3) into the equation for F: Wₘ=M×A×D
5. Maximum Change in Dynamic Acceleration (MCDA) is a mathematical treatment of TEEM data which doesn't change acceleration values or dimensional units.
6. Total Maximum Change in Dynamic Acceleration [MCDA)_{T-Area}] is the sum of the area under each (MCDA) Time (T) curve and is equal to the integral, ∫ yᵢdx, where yᵢ=height of a rectangle segment, (i), with infinitesimal base width, dx. After integration, [(MCDA)_{T-Area}] is equal to (Σyᵢ)(x); or since:
   (Σyᵢ) is proportional to (MCDA) and
   (x) proportional to (T),
   then by substitution: [MCDA)_{T-Area}] is proportional to (MCDA)(T).
7. VO₂ Max is the measured maximum oxygen consumption rate of an individual during an aerobic stress test and is usually expressed as VO₂/M.
   Assumptions:
8. MCDA has the same units and is proportional to acceleration (A).
9. Distance (D) on a treadmill is proportional to Time (T).
10. The product (MCDA)×(T) is proportional to the product (MCDA)×(D) since (D) is proportional to (T).
11. During a VO₂ test, oxygen consumption increases with time in a regular manner until VO₂ Max and can be approximated mathematically as a triangle with the base (B) equal to (time) and the height (H) equal to (oxygen consumption rate). Then the total O₂ consumption is equal to the area of the triangle and the maximum VO₂ Max equals the maximum height of the triangle.
12. During the VO₂ test, total oxygen consumption was calculated from the sum of the average consumption rate for each minute interval. The average oxygen consumption for each minute was calculated by adding the rate at the end of the previous minute to the rate at the end of the present minute and dividing by 2. At the start of the first minute, the standard ‘at rest rate’ of 3.5 ml/min/kg of body weight was used. The amount of O₂ consumed for the last interval was calculated as its factional proportion of a minute, still using the average rate for that interval.
   Resultant Equations:
   Total Work:
13. From (4) above, Mechanical Work(W_{M}) from the TEEM data=[M×A×D]. Substituting the equivalences from (7) & (8) above, we obtain: W_{M} is proportional to [(M)×(MCDA)×(T)].
14. Total Mechanical Work (W_{M})_{T} for the duration of each test=[(M)×(MCDA)×(T)_{T} from (10) above. By substitution from (6) above, (W_{M})_{T} is then proportional to: [(M)×(MCDA)_{T-area}]
15. Biological Work (W_{B}) is proportional to (VO₂) consumed. Total Biological Work (W_{B})_{T} is proportional to Total (VO₂) consumed.
16. Equating (11) to (12) above we get:
   (W_{B})_{T}=W_{M})_{T} or:
   Total (VO₂) consumed is proportional to [(M)×(MCDA)_{T-area}].
   In conventional VO₂ measurements, oxygen consumption is expressed as VO₂/M. Thus, by dividing each side of the proportionality by M, our final relationship is:
   Total (VO₂/M) is proportional to (MCDA)_{T-Area}.
17. A graph of Total (VO₂/M) versus (MCDA)_{T-Area} for all the individuals should be linear and follow the general equation Y=aX+b.
   VO₂ Max:
18. From (11) above based on a triangle's Area=½ BH, where:
   Area=total O₂ consumed
   B=time to VO₂ Max
   H=VO₂ Max, then:
   (Total O₂)=½(Time to VO₂ Max) (VO₂ Max), or
   (VO₂ Max)=[2(Total O₂)/(Time to VO₂ Max)]
19. A graph of (VO₂ Max) versus [2(Total O₂)/(Time to VO₂ Max)] for all the individuals should be linear and follow the general equation Y=aX+b.

### Conclusion

Total Work:

The data of 8 treadmill individuals with a straight-line fit has a correlation coefficient of 0.83.
   VO₂ Max:
The data of 7 treadmill individuals with a straight-line fit has a correlation coefficient of 0.98. One individual was eliminated from data treatment since he was not able to remain on the treadmill for sufficient time to reach VO₂ Max.

### Appendix II

### Treadmill Measured Calorie Expenditure vs. Teem Calculated Calorie Expenditure

Definitions:

1. TEEM=Total Energy Expenditure Measurement
2. Acceleration (A)=Distance/Time²=(D)/(T)² with units in (cm/sec²)
3. Force (F)=Mass×Acceleration=(M)(A) with units in [(g)(G)] or [(g)(cm/sec⁻²)]
4. Work (W)=Energy (E)=Force×Distance=(F)(D) (with units of ergs, calories) by substituting (3) into this equation for (F) we obtain:
   E=(M)(A)(D) with units in [(g)(G)(cm)] or [(g)(cm²/sec²)] 4.1
5. Distance (D) on a treadmill is equal to time (T) of the test multiplied by the treadmill rate (R) thus D=(T)(R) or by substituting for (D) in equation 4.1 we get:
   E=(M)(A)(T)(R) with units in [(g)(G)(cm)] or [(g) (cm²/sec²)] 5.1
6. Maximum Change in Dynamic Acceleration (MCDA) is a mathematical treatment of the TEEM device acceleration data, which measures acceleration values in G's, and is proportional to (A) thus:
   (A)=(α)(MCDA), where: (α) is a proportional constant. Then by substitution for (A) in equation 5.1 we get: 6.1
   E=(M)(α)(MCDA)(T)(R) 6.2
7. VO₂ is the measured oxygen consumption of an individual during an aerobic stress test and is expressed in ml/min or L/min.
   Conversion Factors and Test Conditions:
8. To convert from G's to cm/sec² multiply by 981 (Ref. 2 below)
9. To convert from ergs to kilocalories multiply by 2.39×10⁻¹¹ (Ref. 2)
10. To convert from Liters of O₂ to kilocalories of energy multiply by 4.8 (Ref. 1)
11. Treadmill rate of speed (R) was 13.4 cm/sec
12. Treadmill slope grade was 0.05
13. At rest energy expenditure, E_{R}=1 kcal/kg/hour or ER=1.67×10⁻² kcal/kg/min (Ref. 1)
14. Total oxygen consumption, Total (VO₂), was obtained by summing the amount of oxygen consumed for each minute interval during the test. The amount of O₂ consumed for the last interval, which was usually less than a minute, was calculated by multiplying the fractional portion of a minute times the last interval consumption rate.
   Energy Expenditure Calculation:
15. Total Maximum Change in Dynamic Acceleration α[(MCDA)ₐᵣₑₐ] is the sum of the area under each (α)(MCDA)(T) curve and is equal to the integral, ∫ yᵢdx, where yᵢ=height of a rectangle segment, (i), with infinitesimal base width, dx. After integration, [(MCDA)_{T-Area}] is equal to (εyᵢ)(x) or since: (εyᵢ) is equal to α(MCDA) and (x) is equal to (T), then:
   (α)(MCDA)(T)−(α)[(MCDA)ₐᵣₑₐ]. Where: 15.1
   (α)(MCDA) is measured in G's and time (T) is measured in minutes.
   Then by substituting 15.1 into 6.2 we get the final equation:
      E=(M)(α)[(MCDA)ₐᵣₑₐ](R). 15.2
   Converting from G's, ergs, kg and minutes we get energy in Kilocalories:
      E(in kcal)=(981)(2.39×10⁻¹¹)(60)(10³)(α)(M)(MCDA)ₐᵣₑₐ](R) 15.3
   Dimensional analysis of equation (15.3):
   E(in kcal)=(cm/sec²/G)(kcal/erg)(sec/min)(kg)(g/kg)(G)(min)(cm/sec).
   After unit cancellation (see 4.1 above): E=(g cm²/sec²)(kcal/erg)=kcal: Simplifying (15.3) when (R)=13.4 (cm/sec)(from 11 above) gives:
      E(in kcal)[1.89×10⁻²(α)(M)(MCDA)ₐᵣₑₐ] 15.4
   E is in kcal, (M) is in kg, (α) is unit less, (MCDA)ₐᵣₑₐ is in G's-min
16. Determination of energy expenditure on a treadmill from TEEM data:
Total energy expenditure (ET) on a treadmill for a person of mass (M) is the sum of the rest component (R) plus the horizontal component (H) plus the vertical component (V):
   E_{T}=ΣE_{R}+E_{H}+E_{V} 16.1
For E_{R}:
   From 13 above, E_{R}=(1.67×10⁻² kcal/min)(M) 16.2
   where: (E_{R}) in kcal, (T) in minutes, (M) in kg
For E_{H} & E_{V}:
Energy expenditure for (E_{H}) and (E_{V}) is recorded as TEEM data and can be calculated from (15.4) above taking into account that (E_{V}) requires 18 times more calorie expenditure than (E_{H}) (ref 1).
   E_{H}=(1.89×10⁻²)(α)(M)(MCDA)ₐᵣₑₐ 16.3
The vertical portion of the treadmill is proportional to the percent grade and can be calculated from:

$$\begin{matrix}{E}_{V} = ( 18 ) ( \% grade ) {E}_{H} \\ = ( 18 ) ( \% grade ) [ 1.89 \times {10}^{- 2} ) ( α ) ( M ) {( MCDA )}_{area} ] \\ = ( 18 ) ( 0.05 ) ( 1.89 \times {10}^{- 2} ) ( α ) ( M ) {( MCDA )}_{area} \\ = ( 1.7 \times {10}^{- 2} ) ( α ) ( M ) {( MCDA )}_{area}\end{matrix} \qquad 16.4$$
Equations 16.3 and 16.4 can be combined and simplified to give:
   E_{H}+E_{V}=E_{H+V}=(3.59×10⁻²)(α)(M)(MCDA)ₐᵣₑₐ 16.5
Then the final equation for energy expenditure measurement from the TEEM data:
   E_{T}=ΣE_{R}+E_{H}+E_{V}=ΣE_{R}+E_{H+V}=(1.67×10⁻²)(T)(M)+(3.59×10⁻²)(α)(M)(MCDA)ₐᵣₑₐ 16.6
17. Determination of energy expenditure on a treadmill from oxygen consumption, VO₂:
   E_{T}(ᵢₙ Kcal)=[(ΣVO₂)(4.8 Kcal/L)] where EVO₂ is total VO₂ in liters and 4.8 kcal/L is the conversion factor (obtained from Ref. 1 below). 17.1
18. Energy calculated from the TEEM data should equal the energy determined by oxygen consumption. Thus equating the two equations we get the equation:
   E_{T}(VO₂)=E_{T(TEEM)}=ΣE_{R}+E_{H+V} 18.1
   Thus from 18.1 and 16.6 above:
      (ΣVO₂)(4.8 Kcal/L)=(1.67×10⁻²)(T)(M)+(3.59×10⁻²)(α)(M)(MCDA)ₐᵣₑₐ 18.2

### Conclusion

19. Graphing (ΣVO₂) vs. (M)(MCDA)ₐᵣₑₐ or a rearrangement of terms will give a straight line. A simpler treatment assumes that since total VO₂ is directly proportional to energy, then (MCDA)ₐᵣₑₐ is too since it records all body movement (including breathing). Then energy obtained from VO₂ can be equated to energy obtained from (MCDA)ₐᵣₑₐ to give:
   [(ΣVO₂)×(4.8 Kcal/L)]=(MCDA)ₐᵣₑₐ 19.1
   Then graphing [(ΣVO₂)(4.8 Kcal/L)] Vs (MCDA)ₐᵣₑₐ or a rearrangement of terms will give a straight line.

### REFERENCES

1. Essentials of Cardiopulmonary Exercise Testing, Jonathan Meyers, Ph.D, First Ed., 1996, Human Kinetics—Publishers
2. Handbook of Chemistry and Physics, Robert C. Weast, Ph.D., 60^{th} Edition, CRC Press, Inc., Boca Raton, Fla. 33431

1. A method for determining physical activity by an individual, comprising:
sensing motions at a selected location on the individual aligned substantially along three orthogonal axes;
analyzing the motions sensed along the three orthogonal axes for correlation with motions at the selected location during various physical activities for determining the physical activity of the individual.

2. The method according to claim 1 including forming signals representative of the motions along the three axes;
combining the signals to form a composite signal; and
scaling the composite signal indicative of the amount of exertion associated with the individual's physical activity.

3. The method according to claim 2 including filtering the scaled composite signal to produce time-dependent acceleration data; and
analyzing the time-dependent acceleration data with data indicative of the individual's heart rate during physical activity to provide an output indicative of the individual's health status.

4. A method of analyzing the health conditions of an individual from performance during an interval of physical activity, comprising:
forming outputs indicative of accelerations aligned along three orthogonal axes at a selected location on the individual;
combining the outputs to form a composite output of the accelerations along the three axes;
determining the maximum changes of acceleration over the interval of the physical activity;
analyzing the maximum changes of acceleration with heart rate of the individual over the interval of the physical activity to provide indication of the health condition of the individual.

5. The method according to claim 4 in which the composite output is formed as a vector combination of the accelerations along the three orthogonal axes.

6. The method according to claim 4 including:
determining the physical activity of the individual substantially correlated with the accelerations aligned along the three orthogonal axes;
altering the outputs indicative of the accelerations by scaling factors associated with the determined physical activity; and
determining parameters indicative of the individual's health condition from the altered outputs of the accelerations.

7. The method according to claim 4 including:
determining change in ambient pressure;
determining activity of the individual substantially correlated with the change in ambient pressure and the accelerations aligned along the three orthogonal axes;
altering the outputs indicative of the accelerations by scaling factors associated with the determined physical activity; and
determining parameters indicative of the individual's health condition from the altered outputs of the accelerations.

8. A method for analyzing the health condition of an individual from performance during an interval of physical activity, comprising:
forming outputs indicative of accelerations aligned along three orthogonal axes at a selected location on the individual;
combining dynamic components of the outputs to form a composite output of the dynamic accelerations along the three axes;
filtering the composite output to provide an indication of V0₂, during the interval of physical activity; and
analyzing the indication of V0₂ with the individual's heart rate during the interval of physical activity to provide indication of the individual's health condition.

9. The method according to claim 8 including graphing the indication of V0₂ and heart rate along coordinate graphic axes.

10. Apparatus for determining an individual's health condition from performance of a physical activity, comprising:
means for sensing accelerations at a selected location on the individual aligned substantially along three orthogonal axes;
means for selecting dynamic accelerations from the sensed accelerations;
means for combining the dynamic accelerations to provide a composite means acceleration;
means for altering the composite acceleration according to the individual's physical activity to provide indication of V0₂; and
means responsive to V0₂ and the individual's heart rate during the physical activity to provide indication of the individual's health condition.

11. Apparatus for determining a physical activity of an individual, comprising:
means for sensing accelerations at a selected location on an individual aligned substantially along three orthogonal axes;
means for analyzing the sensed accelerations to provide indication of the physical activity.

12. The apparatus according to claim 11 in which the means for analyzing includes:
means for comparing the sensed accelerations with stored values representative of various physical activities to provide indication of the physical activity for which the sensed and stored acceleration values substantially correspond.

13. A program for implementing computed determination of an individual's physical activity, comprising on a storage medium, a program for implementing alteration of acceleration information sensed substantially along three orthogonal axes at a selected location on the individual to provide dynamic components of the sensed acceleration information, and for analyzing the dynamic components of the sensed acceleration information for correlation with known acceleration information along three orthogonal axes associated with a plurality of physical activities to provide indication of the individual's physical activity.

14. The program according to claim 13 further implementing comparison of the dynamic components of the sensed acceleration information with stored acceleration information from such selected location on an individual aligned along three orthogonal axes associated with the plurality of physical activities to select therefrom the specific physical activity of the individual for which the sensed acceleration information best fits the stored acceleration information.

15. The method according to claim 4 including counting heart beats during an interval of the physical activity; and
logically combining acceleration and count of heart beats to provide an indication of cardiovascular index.

16. The method according to claim 15 in which expended energy during the interval of physical activity is determined from the acceleration; and
the cardiovascular index is determined as a ratio of expended energy to the count of heart beats.
